(19) United States
(12) Patent Application Publication
(10) Pub. No.: US 2019/0175858 A1
(43) Pub. Date: Jun. 13, 2019

(54) DEVICES AND METHODS FOR NON-INVASIVE CARDIO-ADAPTIVE POSITIVE PRESSURE VENTILATION THERAPY

(71) Applicant: SomnaCardia Inc., Mountain View, CA (US)

(72) Inventors: Maria A. Parfenova, Los Altos, CA (US); Alexandr S. Parfenov, Moscow (RU)

(21) Appl. No.: 16/214,426

(22) Filed: Dec. 10, 2018

Related U.S. Application Data
(60) Provisional application No. 62/596,885, filed on Dec. 10, 2017.

(51) Int. Cl. A61M 16/00; A61B 5/024; A61B 5/021; A61B 5/0245; A61B 5/11; A61B 5/0295; A61B 7/04; A61B 5/029; A61B 8/08; A61B 5/00; A61B 5/08; A61M 16/06; A61M 16/08

(52) U.S. Cl. CPC A61M 16/026; A61M 2205/3569; A61B 5/02116; A61B 5/0245; A61B 5/1102; A61B 5/0295; A61B 7/04; A61B 5/029; A61B 8/0883; A61B 5/4035; A61B 5/02405; A61B 5/08; A61M 16/0057; A61M 16/06; A61M 16/0875; A61M 2016/0027; A61M 2205/3303; A61B 5/02416

(57) ABSTRACT

In one embodiment, a cardio-adaptive non-invasive positive airway pressure device comprises an airflow generator to provide pressurized air to a human. A detector detects a cardiac cycle of the human. A control unit estimates a next cardiac cycle based on the detected cardiac cycle and provides a control signal to the air flow generator to control timing of the providing of the pressurized air to the human based on the estimated cardiac cycle.

### RELATED APPLICATIONS

This application claims the benefit under 35 USC § 119 to U.S. Provisional Patent Application Ser. No. 62/596,885 filed on Dec. 10, 2017, which is incorporated by reference herein in its entirety.

### FIELD

This invention relates generally to the non-invasive ventilation devices; and, more particularly to improving the safety and effectiveness of non-invasive positive pressure ventilation devices on cardiovascular health.

### BACKGROUND

Unless otherwise indicated herein, the approaches described in this section are not admitted to be prior art by inclusion in this section.

Non-invasive ventilation (NIV) can be defined as a ventilation modality that supports breathing by delivering mechanically assisted breaths without the need for intubation of or surgical access to airway. NIV devices are used extensively to treat acute and chronic conditions in hospital setting and at home. NIV is sub-divided into negative pressure ventilation and non-invasive positive pressure ventilation (NPPV). NPPV primarily refers to positive airway pressure (PAP) devices. The existing NPPV therapies are used to treat a number of conditions, with therapies for acute respiratory failure and sleep apnea being the main applications. The existing NPPV therapies also are used to treat congestive heart failure as a supplementary treatment.

Sleep apnea (SA) and congestive heart failure (HF) are highly prevalent disorders and are responsible for high morbidity and mortality.

SA is characterized by recurrent failures to breathe adequately during sleep (termed apneas or hypopneas) as a result of obstructions in the upper airway or a failure to generate sufficient respiratory effort. Apnea is defined as a complete cessation of airflow. Hypopnea is defined as a reduction in airflow disproportionate to the amount of respiratory effort expended and insufficient to meet the individual's metabolic needs. During apnea or hypopnea, commonly referred to as abnormal respiratory events, oxygen levels in the brain decrease, while the carbon dioxide levels rise, causing the sleeper to awaken. During an apneic event, the sympathetic nerve tone increases, adrenaline and cortisol are released into blood and the heart rate and blood pressure increase. The brief arousals to breathe are followed by a return to sleep. Untreated SA patients are three to five times more likely to be involved in industrial and motor vehicle accidents and have impaired vigilance and memory. Untreated SA leads to hypertension, stroke, heart failure, irregular heartbeat, heart attack, diabetes and depression.

The most common approach to treating SA is to supply a patient with positive airway pressure (PAP) via an NPPV device comprised of a pump or a flow generator, a hose and a mask. PAP therapy and its various forms (continuous, bilevel, adaptive servo-ventilation, auto-, etc.) provide an air pressure “splint” in order to keep the airway open and to pump air into lungs.

PAP therapy has been proven effective in improving and even reversing some of the negative impact of SA on health. Treatment with PAP has been shown to decrease daytime sleepiness, depression and high blood pressure.

Heart failure (HF) develops when the heart muscle's function as a pump fails to meet the body's needs. With HF, blood moves through the heart and body at a slower rate, and pressure in the heart increases. As a result, the heart cannot pump enough oxygen and nutrients to meet the body's needs. The chambers of the heart may respond by stretching to hold more blood to pump through the body or by becoming stiff and thickened. This helps to keep the blood moving, but the heart muscle walls may eventually weaken and become unable to pump as efficiently. As a result, the kidneys may respond by causing the body to retain water and salt. If fluid builds up in the arms, legs, ankles, feet, lungs, or other organs, the body becomes congested, and congestive heart failure is the term used to describe the condition.

NPPV therapy is used to supplement the pharmacological approaches to treat HF. In the wide spectrum of HF care, NPPV therapy is used to reduce pulmonary edema, improve oxygenation, reduce cardiac load, improve lung and respiratory muscle function, alleviate hypoventilation and hypercapnia, and normalize abnormal respiratory patterns, such as Cheyne-Stokes respiration.

While the NPPV therapy is overall beneficial to HF and SA patients and is generally believed to be safe, the effects of NPPV on cardiovascular system are both positive and negative. On the positive side, in patients with SA, the NPPV can reduce elevated blood pressure, improve arrhythmia, increase blood oxygenation and reduce the heightened level of sympathetic nervous activity. On the negative side, the NPPV therapy exerts a host of effects that impair key cardiovascular functions. The additional pressure generated by NPPV in the thoracic cavity compresses the heart and the large blood vessels. Specifically, venous return is reduced due to compression of superior vena cava and other large veins. Under the additional pressure from an NPPV device the heart may not expand as much during diastole. As a result, the cardiac output and ejection fractions of the heart are reduced. In SA patients, venous return and cardiac output (both are measures of the cardiovascular system performance) are acutely reduced by as much as 30% each within minutes of initiating an NPPV. It has also been shown that an NPPV affects the cranial blood flow and cerebrospinal fluid circulation.

The right heart (i.e., right atrium and right ventricle) functions at pressures much lower than those in the left heart. Commonly prescribed NPPV pressures (10-20 cmH2O) are comparable to those in the right heart and pulmonary artery. Therefore right heart is especially susceptible to the effects of NPPV. During an NPPV a decreased preload in the right heart is coupled with an increased afterload due to the compression of the pulmonary arteries. In fact clinically significant pulmonary hypertension resulting from SA often persists in patients on PAP therapy. Because the cardiovascular system is a closed circuit system, the decreased cardiac output of the right heart results in a decreased cardiac output of the left heart within several cardiac cycles. Furthermore, by compressing the pericardium NPPV can decrease the blood flow through the coronary arteries and therefore deprive the myocardium of oxygen and nutrients and therefore further impair the heart function.

In HF patients, NPPV has several effects on hemodynamics: first, NPPV diminishes systemic venous return and right ventricle (RV) preload by increasing intrathoracic pressure. Second, NPPV alters the pulmonary vascular resistance (PVR), which is the major determinant of RV afterload, via an alteration in lung volume. The short-term application of PAP (for example, a pressure of 5-10 cm H2O) can increase cardiac output in stable HF patients with pulmonary congestion. However, the response of PVR to an increasing PAP follows a U-shaped curve and is patient specific due to lung volume variation (i.e., the lowest PVR can be observed with a lung volume around functional residual capacity). Since PAP can increase RV afterload, it is of significant concern in HF patients and can limit the use of NPPV. Therefore, a therapeutic approach that combines the beneficial effects of NPPV without its negative impact on the RV afterload has the potential to become an important and safe tool in proactive management of hemodynamic congestion.

The negative effects of NPPV in all patients can be reduced if the air pressure is delivered in a manner in which it minimally interferes with the normal functioning of the heart and the blood flow.

Moreover, NPPV can assist the cardiovascular system if the pressure to the lungs is delivered in a manner that assists the blood flow through the central blood vessels in the thoracic cavity and the heart.

Currently, there are several approaches to reducing the overall pressure provided by NPPV. Originally NPPV was administered as continuous PAP therapy (CPAP) which provides a flow of air at a pre-set pressure. BiPAP (a bi-level PAP) was developed to reduce the pressure during exhalation. AutoPAP (a form of CPAP) adjusts the overall pressure to better match the needs of a patient in real time and to avoid overpressurizing the patient. Adaptive servo-ventilation (ASV, a form of BiPAP) delivers air pressure only when apneas are detected.

While these existing modifications temporarily and partially relieve pressure during PAP therapy, these pressure reductions are timed to the breathing patterns. Each breath cycle occurs over a period of several heart beats (for instance approximately 4 heart beats per breath in a healthy person at rest). So, even when the pressure is relieved during exhalation in a BiPAP therapy, at least two cardiac cycles out of four are still significantly affected by the increased intrathoracic pressure due to high PAP during the inhalation. In summary, no existing form of PAP therapy eliminates the negative effects of the increased intrathoracic pressure on the cardiovascular system.

### SUMMARY

The invention described herein is a form of NPPV and improves upon the existing devices and methods. While the described invention is applicable for treatment of any condition that can be treated with the existing NPPV therapies, the described invention can also be used for additional indications such as treatment of worsening congestive heart failure and improvement of hemodynamics during septic shock. These additional applications of the described invention are feasible due to the fact that the invention can assist the cardiovascular system in ways the existing NPPV therapies cannot. The invention is described specifically for addressing the shortcomings of the NPPV for the treatment of sleep apnea and for providing the therapeutic benefits beyond those currently available with NPPV for the treatment of worsening congestive heart failure.

In some embodiments, a modification to the existing NPPV devices and methods where the pressure relief is timed to a specific portion of the cardiac cycle can therefore reduce the negative effect of the NPPV on cardiovascular health. Furthermore, a modification to the existing NPPV devices where the peak pressure is timed to a specific potion of the cardiac cycle such as the systole of the atria for instance would assist the heart in its contractile function.

In a general aspect, the present invention relates to a non-invasive positive pressure ventilation (NPPV) device for treating various conditions such as congestive heart failure (HF), acute respiratory failure, sleep apnea (SA) and improvement of hemodynamics during septic shock.

The device includes a pump or a flow generator, a unit that identifies the cardiac cycles in a patient and a control unit that times pressure levels generated by the pump to the cardiac cycles as to minimize the negative effects of increased intrathoracic pressure on the functioning of the cardiovascular system.

In another general aspect, the present invention relates to a computer program which detects cardiac cycles from a sensor and modifies the pressure from a PAP device.

Implementations of the device may include one or more of the following. The cardiac cycles of a patient may be detected with different types of sensors: electrodes, arterial pressure sensors, photo-sensors (such as those utilized in pulse oximeters), electro-magnetic, acoustic, accelerometry, ballistography, plethysmography sensors or contactless sensors, for instance. The NPPV pressure relief as well as the peak PAP during the cardiac cycle can be achieved by including one of the following: a control unit that changes the operation of the pump, and optionally a valve (or a combination of valves) that does not affect the pump but alters the airflow from the pump.

The profile of the PAP pressure variation during the cardiac cycle may be represented by a sine wave or a wave of a complex shape tailored to providing pressures which minimally disrupt or even enhance the blood flow through the blood vessels in the thoracic cavity and the heart.

Implementations of the device may include one of the following. A complete NPPV system delivering air pressure timed to cardiac cycles or an add-on unit to the existing NPPV systems or a computer program that changes the operation of an existing NPPV system.

Implementations of the devices may include a calibration algorithm or system for identifying the timing of airway pressure profile for each patient since the airway size, lung size and lung compliance vary among patients. A calibration can be used once for each patient or once at the beginning of each therapy session, or periodically throughout a therapy session.

The described devices and methods provide an improvement to the NPPV in order to minimize the negative effects on cardiovascular system. Furthermore, the described devices assist the blood flow through the thoracic cavity and the heart. The described devices and methods are therefore safer and more beneficial to the patient's cardiovascular health than the other known NPPV techniques.

The present disclosure provides a cardio-adaptive non-invasive positive airway pressure device. An airflow generator provides pressurized air to a human. A detector detects a cardiac cycle of the human A control unit estimates a next cardiac cycle based on the detected cardiac cycle and provides a control signal to the air flow generator to control timing of the providing of the pressurized air to the human based on the estimated cardiac cycle.

In one embodiment, the control unit selects a reference point in the detected cardiac cycle, determines a duration of a plurality of detected cardiac cycles, estimates a reference point for a next cardiac cycle, and sets the estimated reference point as a timing parameter for the control signal.

In one embodiment, the control unit sets the timing parameters so that a pressure trough of the pressurized air substantially occurs with the estimated reference point.

In one embodiment, the cardio-adaptive non-invasive positive airway pressure device further comprises a valve timing the delivery of the provided pressurized air to the next cardiac cycle.

In one embodiment, the detector of a cardiac cycle is selected from a group of an arterial volume detection system (a photoplethysmography or a plethysmography system), an arterial pressure pulse wave detection system, an electrocardiography system, an acoustic heart beat detection system, and a ballistic heart beat detection system.

In one embodiment, the detector of a cardiac cycle is a remote system that is not in direct contact with the patient.

The present disclosure provides a method for determining cardio-adaptive positive airway pressure. The method comprises detecting cardiac cycle of a human; estimating a next cardiac cycle; and determining timing parameters of pressurized air based on the estimated cardiac cycle.

In one embodiment, the method further comprises providing pressurized air to the human according to the timing parameters.

In one embodiment, estimating a next cardiac cycle comprises selecting a reference point in the detected cardiac cycle; determining a duration of a plurality of detected cardiac cycles; and estimating a reference point for a next cardiac cycle; and determining timing parameters of pressurized air based on the estimated cardiac cycle comprises setting the estimated reference point for the timing parameters for providing pressurized air to the human.

In one embodiment, the method further comprises providing pressurized air to the human according to the timing parameters so that a pressure trough of the pressurized air substantially occurs with the estimated reference point.

In one embodiment, the method further comprises periodically adjusting the timing parameters of the pressurized air during the treatment period.

In one embodiment, the method further comprises detecting one or more physiological parameters before pressurized air is delivered to the human; delivering, during an initial time period, pressurized air having a cardio-adaptive airway pressure profile to the human; detecting one or more physiological parameters during the initial time period; comparing one or more physiological parameters before and during the initial time period; adjusting the parameters of the airway pressure profile; and delivering the pressurized air having the adjusted airway pressure profile to the human.

In one embodiment, the detected physiological parameter is selected from a group of systemic arterial pressure, pulmonary arterial pressure, cardiac output, systemic vascular resistance, and pulmonary vascular resistance.

In one embodiment, the detected physiological parameters are selected from a group of contour characteristics of the arterial pulse wave, variability of arterial pulse wave amplitude, shape characteristics of the echocardiogram, autonomous nervous system status, frequency components of the heart rate variability frequency spectrum, and the frequency components of the arterial pulse wave frequency spectrum.

In one embodiment, contour characteristics of an arterial pulse wave is an augmentation index.

In one embodiment, contour characteristics of an arterial pulse wave is the maximal slope of the ascending systolic portion or the descending diastolic portion of the pulse wave.

In one embodiment, the shape characteristic of electrocardiogram or the contour characteristic of arterial pulse wave is a ratio of the duration of systole to the duration of diastole.

In one embodiment, detecting cardiac cycle of a human comprises detecting airway pressure; and determining cardiac cycle based on the detected airway pressure.

The present disclosure provides a method for determining a timing parameter of pressurized air. The method comprises selecting a reference point in a detected cardiac cycle; determining a duration of a plurality of detected cardiac cycles; and estimating a reference point for a next cardiac cycle for setting the timing parameter to provide pressurized air to the human.

In another embodiment, the present invention includes a computer readable medium embodying a computer program for performing methods and embodiments described herein.

In another embodiment, the present invention includes a computer system comprising one or more processors implementing the techniques described herein.

Although the invention has been particularly shown and described with reference to multiple embodiments, it will be understood by persons skilled in the relevant art that various changes in form and details can be made therein without departing from the spirit and scope of the invention.

The following detailed description and accompanying drawings provide a better understanding of the nature and advantages of the present disclosure.

### BRIEF DESCRIPTION OF THE DRAWINGS

With respect to the discussion to follow and in particular to the drawings, it is stressed that the particulars shown represent examples for purposes of illustrative discussion, and are presented in the cause of providing a description of principles and conceptual aspects of the present disclosure. In this regard, no attempt is made to show implementation details beyond what is needed for a fundamental understanding of the present disclosure. The discussion to follow, in conjunction with the drawings, make apparent to those of skill in the art how embodiments in accordance with the present disclosure may be practiced. In the accompanying drawings:

The following drawings, which are incorporated in and form a part of the specification, illustrate embodiments of the present invention and, together with the description, serve to explain the principles of the invention.

FIG. 1 is a drawing illustrating one embodiment of a cardio-adaptive positive airway pressure system including a sensor for cardiac cycle detection that is incorporated into a mask and is connected via a wire with a main unit.

FIG. 2 is a drawing illustrating an embodiment of a cardio-adaptive positive airway pressure system including a contactless sensor for cardiac cycle detection that transmits its data wirelessly to a main unit.

FIG. 3 is a drawing illustrating an embodiment of a cardio-adaptive positive airway pressure system including a sensor for cardiac cycle detection that is incorporated into a watch and transmits its data wirelessly to a main unit.

FIG. 4 is a drawing illustrating an embodiment of a cardio-adaptive positive airway pressure system including a sensor for cardiac cycle detection that is incorporated into a ring and transmits its data wirelessly to a main unit.

FIG. 5 is a drawing illustrating an embodiment of a cardio-adaptive positive airway pressure system including three electrocardiogram (ECG sensors) that detect cardiac cycle and a volume-clamp device that detects beat-to-beat blood pressure.

FIG. 6 is a timing diagram illustrating an ECG cardiac cycle and the periods during which an increase in intra-thoracic pressure negatively impacts the functioning of the heart.

FIG. 7 is a schematic diagram illustrating effects of PAP on the right heart and large blood vessel in the thoracic cavity.

FIG. 8 is a block diagram illustrating the main components of a cardio-adaptive positive airway pressure system.

FIG. 9 is a timing diagram illustrating the timing of the negative impact of bi-level PAP on the heart and large blood vessel in the thoracic cavity; and one example of the timing of the cardio-adaptive PAP pressure relief troughs.

FIG. 10 is a flow diagram of a method for determining timing parameters of pressurized air based on the detected cardiac cycles. This figure also includes waveforms for two examples of this method when the cardiac cycles are detected with ECG and PPG sensors.

FIG. 11 is a flow diagram of the method for adjusting the timing parameters of cardio-adaptive PAP.

FIG. 12 is a graph illustrating the effect of the phase shift of the pressure relief troughs of cardio-adaptive PAP on beat-to-beat systolic blood pressure.

FIG. 13 is a graph illustrating the acute effects of continuous PAP and cardio-adaptive PAP on augmentation index of arterial pulse wave.

FIG. 14 is a graph illustrating the overnight effect of continuous PAP and cardio-adaptive PAP therapy on nocturnal systolic blood pressure.

### DETAILED DESCRIPTION

In the following description, for purposes of explanation, numerous examples and specific details are set forth in order to provide a thorough understanding of the present disclosure. It will be evident, however, to one skilled in the art that the present disclosure as expressed in the claims may include some or all of the features in these examples, alone or in combination with other features described below, and may further include modifications and equivalents of the features and concepts described herein.

Reference in the specification to “one embodiment”, “an embodiment”, “various embodiments” or “some embodiments” means that a particular feature, structure, or characteristic described in connection with these embodiments is included in at least one embodiment of the invention, and such references in various places in the specification are not necessarily all referring to the same embodiment.

The term “patient” is used herein for convenience. The term “human” may be used interchangeably with the term “patient.”

Augmentation of arterial pulse wave represents the difference between the second and first systolic peaks of an arterial pulse waveform, and the augmentation index represents the augmentation expressed as a percentage of the pulse wave amplitude.

The term “pressure profile” is used interchangeably with the term “pressurized air having a pressure profile.”

The maximal slope of the ascending systolic portion of the arterial pulse wave is reflective of the contractile force generated by the left ventricle.

The maximal slope of the descending diastolic portion of the arterial pulse wave is reflective of the systemic vascular resistance.

The ratio of the durations of the systolic and the diastolic portions of the arterial pulse wave or an electrocardiogram is reflective of the contractile force of the myocardium.

Because the direct measures of the hemodynamics (such as cardiac output, systemic and pulmonary vascular resistance and the systemic, pulmonary and central venous pressure) are invasive or difficult or expensive to perform, the indirect measures (such as the above described augmentation index, maximal slopes of the arterial pulse wave and the ratio of the durations) can be used to determine the effect of an NPPV treatment on hemodynamics.

Cardio-adaptive positive airway pressure systems provide air with variable air pressure to a patient with timing based on the specific cardiac cycles or physiological parameters of the patient. In some embodiments, the cardio-adaptive positive airway pressure system estimates a reference point based on the cardiac cycles or physiological parameters of the patient, and uses the estimated reference point for the timing of variations in air pressure to control the impact on the cardiovascular system of the patient.

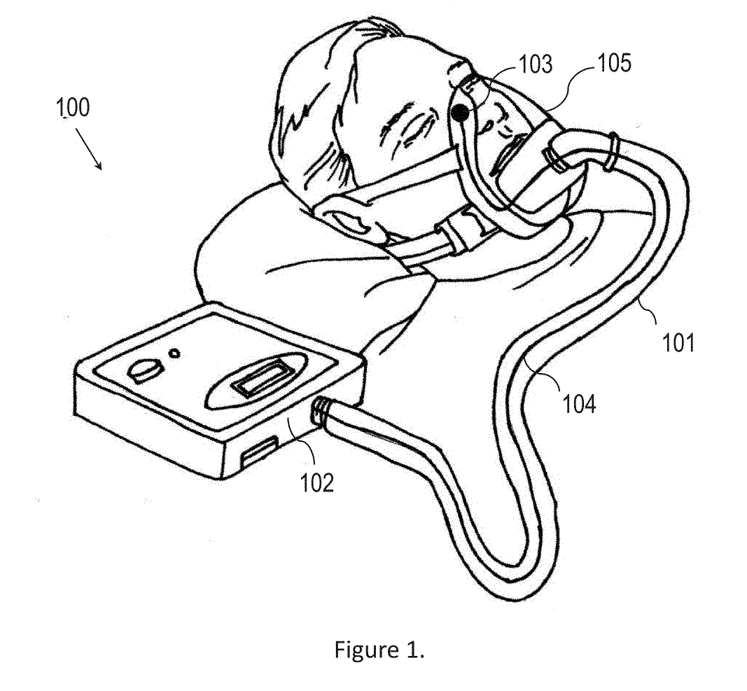

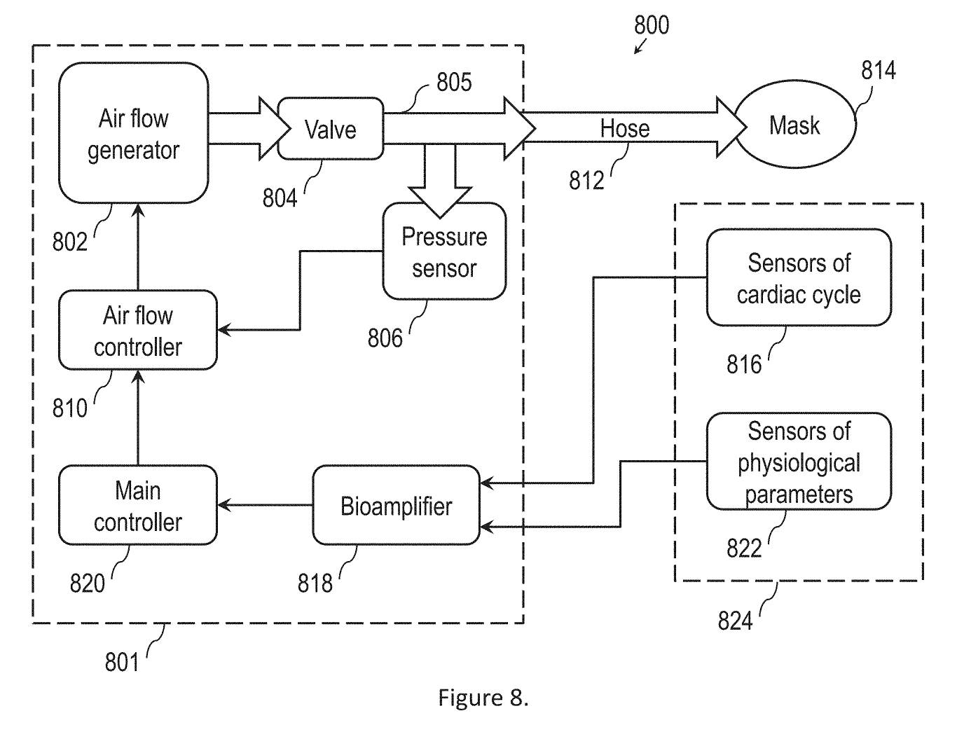

Referring to FIG. 1, an illustration of one embodiment of a cardio-adaptive positive airway pressure system 100 is shown. The cardio-adaptive positive airway pressure system 100 detects cardiac cycles, estimates a reference point from the cardiac cycles for the next cycle, and provides air pressure to the patient based on the estimated reference point. The cardio-adaptive positive airway pressure system 100 may be the cardio-adaptive positive airway pressure system described below in conjunction with FIG. 8. A full face mask 105 is attached to the patient. A hose 101 connects to the mask 105 and a main unit 102 that includes an air flow generator. A photosensor 103 is incorporated into the seal of the mask 105 and is in direct contact with the skin of the patient. The photosensor 103 is powered and transmits its signal via a wire 104. The photosensor 103 detects cardiac cycles of the patient and one or more physiological parameters.

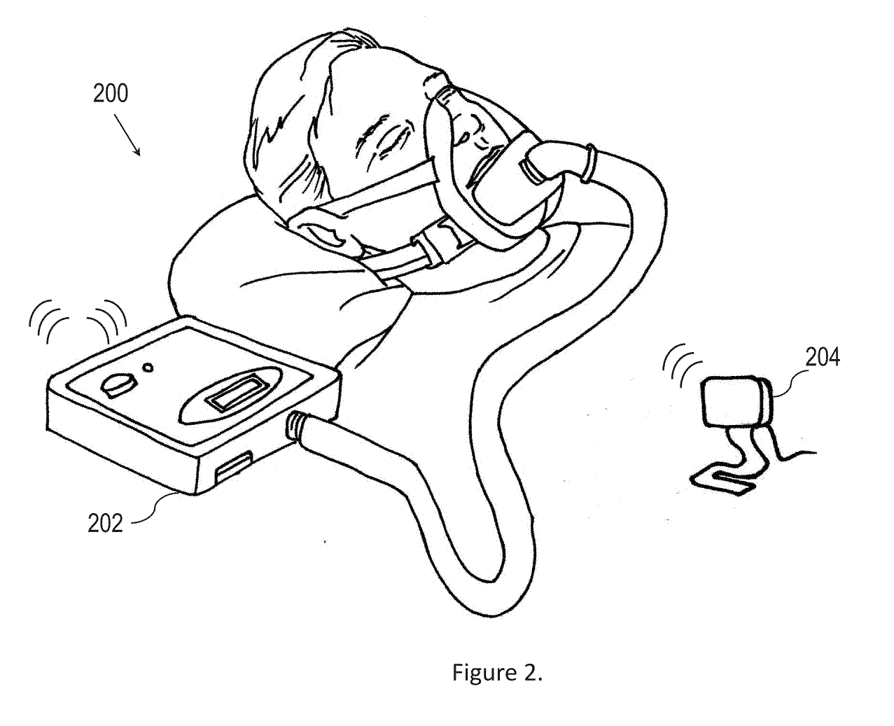

Referring to FIG. 2, an illustration of one embodiment of a cardio-adaptive positive airway pressure system 200 is shown. System 200 is similar to the system 100 (FIG. 1), but includes one or more contactless sensors 204 to remotely detect cardiac cycles and one or more physiological parameters of the patient, and a main unit 202 instead of a main unit 102. The main unit 202 communicates, such as wirelessly, with the contactless sensors 204. The sensors 204 are not integrated into the mask, but rather located externally for remote contactless sensing. The sensors 204 may be, for instance, light-reflective cameras, or radio frequency systems. The remote sensors 204 can be integrated into the main unit 202. The cardio-adaptive positive airway pressure system 200 may be the cardio-adaptive positive airway pressure system described below in conjunction with FIG. 8.

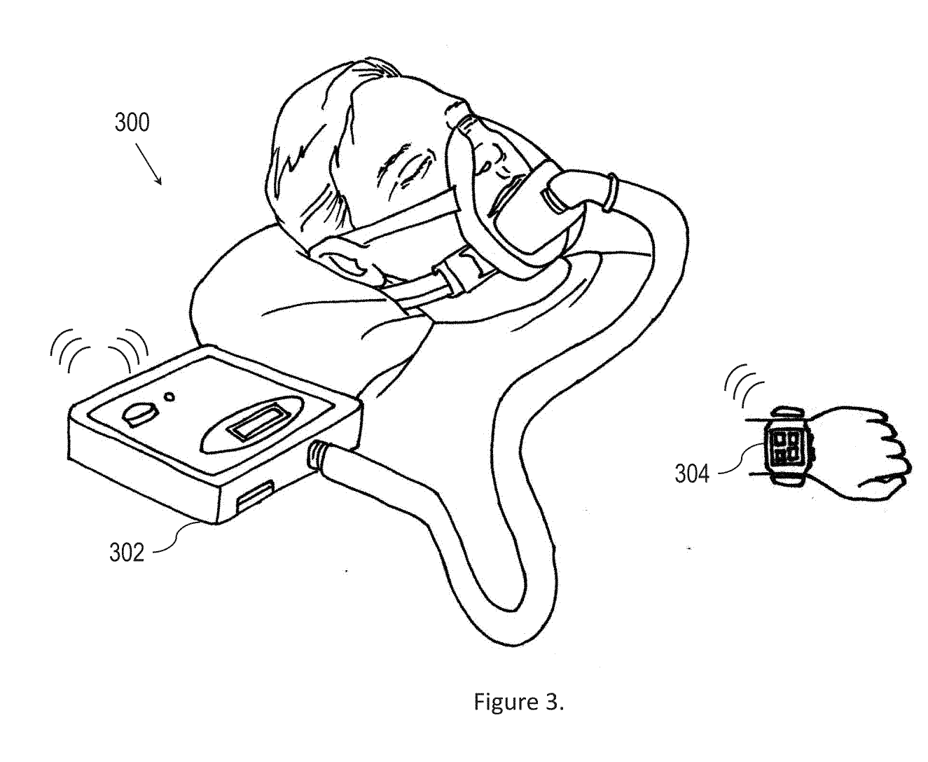

Referring to FIG. 3, an illustration of one embodiment of a cardio-adaptive positive airway pressure system 300 is shown. System 300 is similar to the system 200 (FIG. 2), but includes a wrist sensor 304 to detect cardiac cycles and one or more physiological parameters of the patient, and a main unit 302 instead of a main unit 202. The main unit 302 communicates, such as wirelessly, with the wrist sensor 304. The sensors 304 are not integrated into the mask, but rather located on the wrist, or integrated into a watch or other wearable for sensing. The sensors 304 may be, for instance, photo-sensors, or pressure sensors. The cardio-adaptive positive airway pressure system 300 may be the cardio-adaptive positive airway pressure system described below in conjunction with FIG. 8.

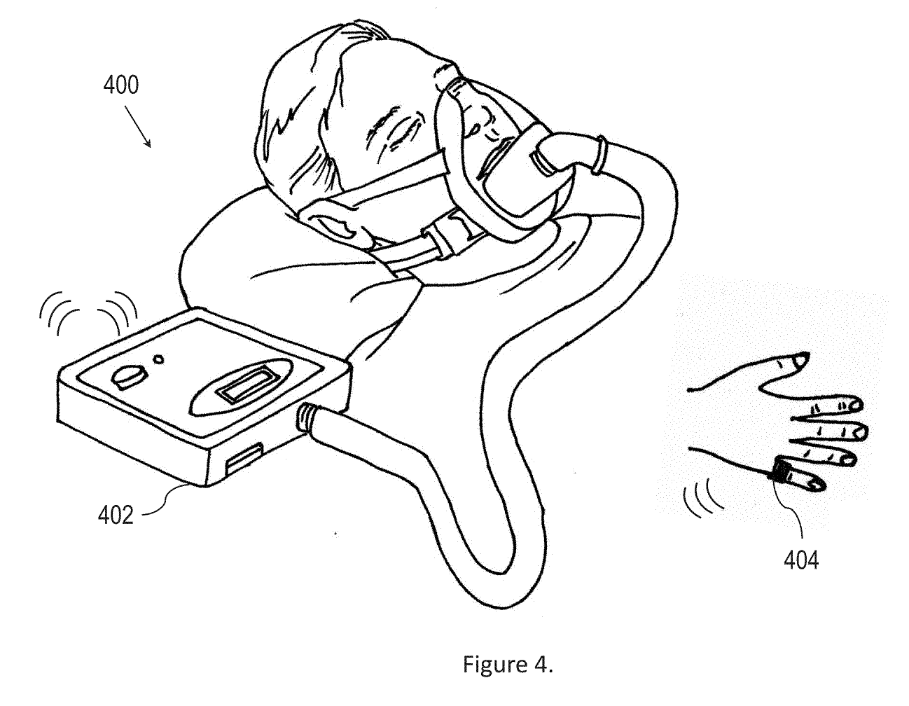

Referring to FIG. 4, an illustration of one embodiment of a cardio-adaptive positive airway pressure system 400 is shown. System 400 is similar to the system 300 (FIG. 3), but includes a finger sensor 404 to detect cardiac cycles and one or more physiological parameters of the patient instead of a wrist sensor 304, and a main unit 402 instead of a main unit 302. The main unit 402 communicates, such as wirelessly, with the finger sensor 404. The sensors 404 are not integrated into the mask, but rather located on the finger, or integrated into a ring or other wearable for sensing. The sensors 404 may be, for instance, photo-sensors, or pressure sensors. The cardio-adaptive positive airway pressure system 400 may be the cardio-adaptive positive airway pressure system described below in conjunction with FIG. 8.

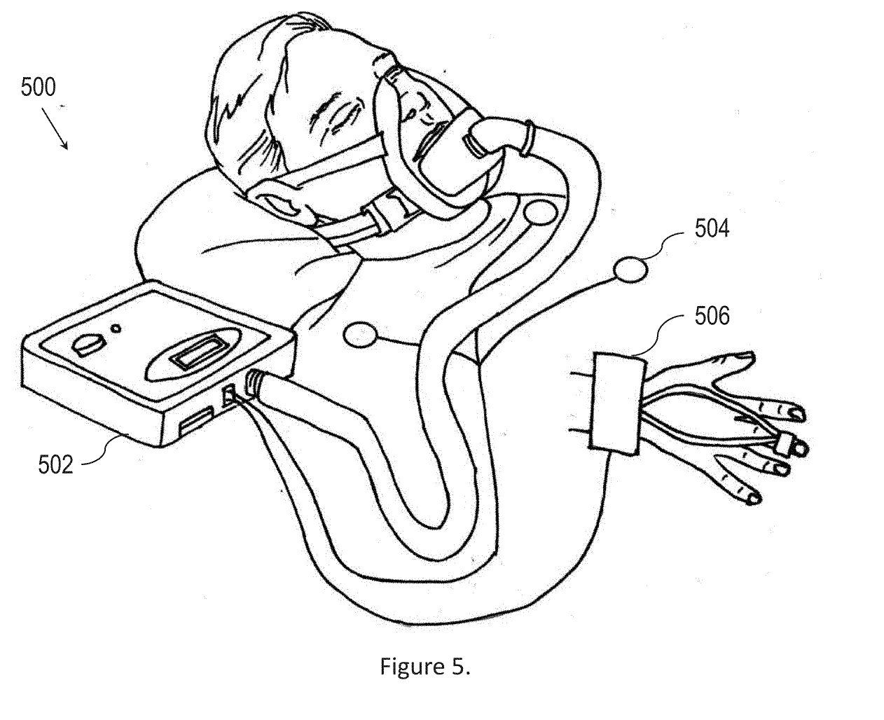

Referring to FIG. 5, an illustration of an embodiment of a cardio-adaptive positive airway pressure system 500 used for conducting the initial clinical testing is shown. The cardio-adaptive positive airway pressure system 500 includes three ECG 504 electrodes to detect ECG signal and to transmit the detected ECG signal to a main unit 502. The system 500 includes a volume-clamp system 506 to detect beat-to-beat systemic blood pressure to evaluate the effect of the cardio-adaptive PAP on the hemodynamics. The cardio-adaptive positive airway pressure system 500 may be the cardio-adaptive positive airway pressure system described below in conjunction with FIG. 8.

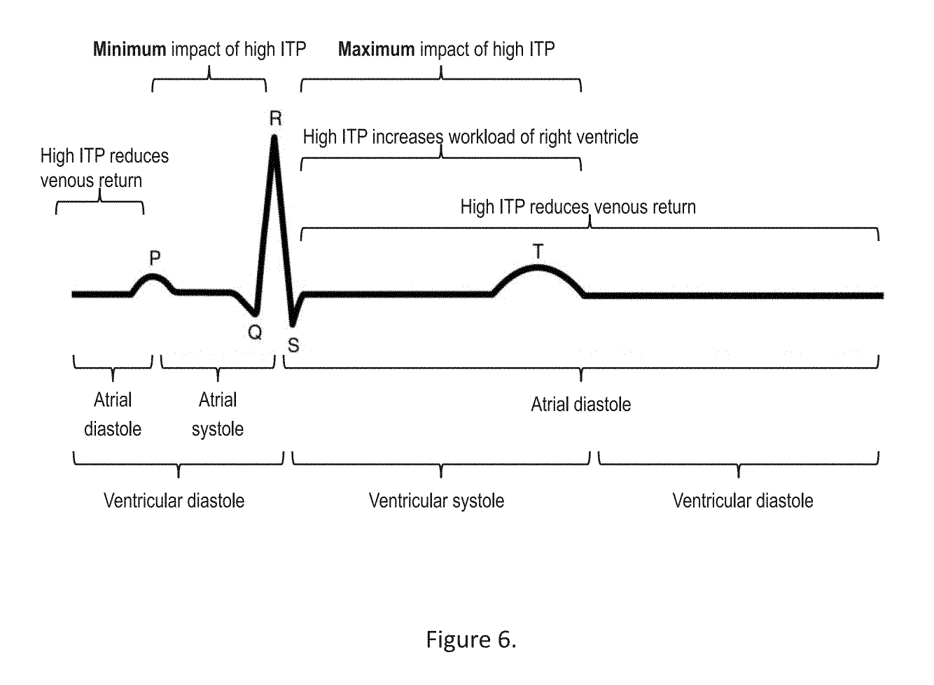

Referring to FIG. 6, an exemplified electrocardiography (ECG) trace of a single heart beat is shown. The timing of the systole (contraction) and diastole (relaxation) of the four heart chambers (two atria and two ventricles) is aligned with the ECG trace. P-wave represents atrial depolarization. The PR-interval represents the time taken for electrical activity to move between the atria and ventricles. The QRS-complex represents depolarization of the ventricles and is seen as three closely related waves on the ECG (Q, R and S wave). The ST-segment is an isoelectric line that represents the time between depolarization and repolarization of the ventricles (i.e. contraction). The T-wave represents ventricular repolarization. The QT-interval represents the time taken for the ventricles to depolarize and then repolarize. The figure also shows the time periods during which high intrathoracic pressure (ITP) impacts venous return and the workload of the right ventricle. The figure identifies the time periods of maximum and minimum impact of high ITP on the cardiovascular system. The maximum negative impact of high ITP on the heart roughly coincides with the systole of the ventricles. The minimum negative impact of high ITP roughly coincides with atrial systole.

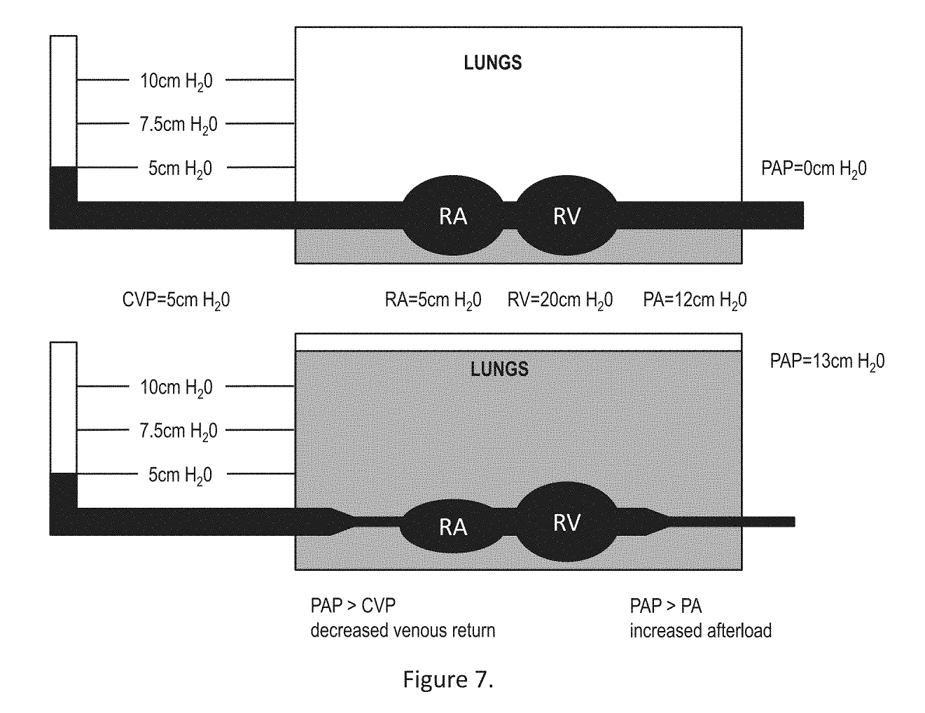

Referring to FIG. 7, the pressures in the central veins and the right heart are shown in comparison with the pressure inside the chest cavity (i.e., intrathoracic pressure). When no positive airway pressure is delivered externally, the intrathoracic pressure during normal breathing does not significantly impact either the venous return or the pulmonary artery (PA) pressure, and therefore the workload of the right atrium (RA) and right ventricle (RV) are unaffected. When positive airway pressure is delivered with an NPPV device, the PAP exceeds central venous pressure (CVP), the venous return is diminished, and the preload is reduced. More importantly the pulmonary arteries are compressed and hence the right ventricle has an increased afterload. As a result, the right ventricle has to work harder and cardiac output is reduced.

Referring to FIG. 8, an illustration of one embodiment of a cardio-adaptive positive airway pressure system 800. The system 800 comprises an air pressure system 801, a mask 814, and a detection system 824. The mask 814 and a detection system 824 are attached to a patient. The detection system 824 may be remote from the patient, such as the system described above in conjunction with FIG. 2. The air pressure system 801 provides cardio-adaptive positive airway pressure via a hose 812 to the mask 814 for providing the air to a patient. The air pressure system 801 comprises an air flow generator 802, an optional valve 804, a pressure sensor 806, an air flow controller 810, a bioamplifier 818, and a main controller 820. The detection system 824 monitors and detects physiological characteristics of a patient. The detection system 824 includes sensors 816 for detecting cardiac cycle and includes sensors 822 for detecting physiological parameters. The detection system 824 may include, for example, ECG electrodes, pressure sensors, impedance sensors, photosensors, acoustic sensors, accelerometry sensors, or any combination thereof. In one embodiment, the detection system 824 comprises a plurality of ECG electrodes (for example, three ECG electrodes) that are applied to a patient and an ECG trace is recorded by the main controller 820. For instance, a photosensor can be incorporated into the flexible seal of the mask 814 which comes into direct contact with the skin (for example, on the bridge or sides of the nose). In some embodiments, the detection system 824 may be integral with a wearable, such as a ring or a watch that is attached to the patient somewhere other than the face of the patient (see, for example, FIGS. 3-5). In some embodiments, the detection system 824 is a contactless sensor (see, for example, FIG. 2). When the detection system 824 or some sensors thereof, is not incorporated into the mask 814, a wireless communication system can be employed to connect to the bioamplifier 818.

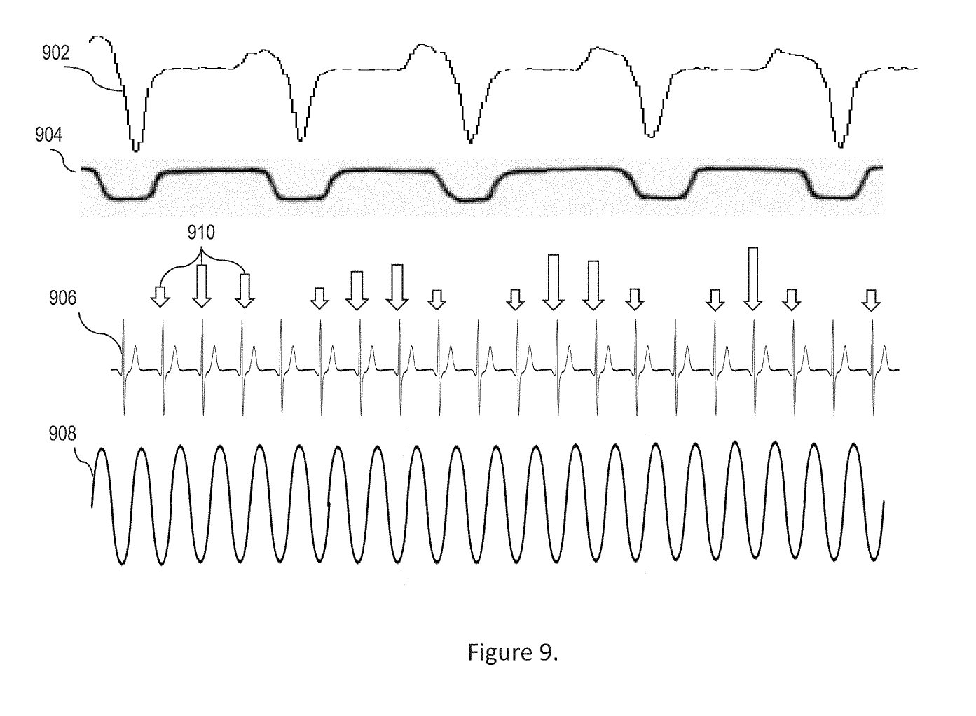

The main controller 820 may perform the processes described below in conjunction with FIGS. 9-11, or may include an automatic R-peak detection software algorithm that generates reference points or a synchronization signal or a trigger signal corresponding to each R-peak. The processes described as being performed by the main controller 820 may be performed, in whole or in part, by processors, computer, servers, and the like that are remote from the air pressure system 801.

The air flow generator 802 includes an air pump (not shown). The air flow generator 802 may include a conventional CPAP system that provides the air flow that air flow controller 810 controls using a valve 804 that regulates the air flow provided to the mask 814. The valve 804 is capable of rapidly varying the pressure levels. A pressure sensor 806 is coupled to the output of the valve 804 and provides output pressure for the air flow controller 810 to regulate the air flow generator 802.

In some embodiments the pressure sensor 806 is used to detect cardiac cycles by detecting airway pressure changes in the tubing 805 connecting the output of the air flow generator 802 and the hose 812. Because the heart is surrounded by the lungs, heart muscle contractions exert pressure on the lungs and these relatively small pressure oscillations can nevertheless be detected in the airway system, the mask 814, the hose 812 or the tubing 805. When pressure sensor 806 is used to detect cardiac cycles it serves as the detection system 824 and is connected to the bioamplifier 818 or the main controller 820. The main controller 820 determines cardiac cycles from the detected airway pressure changes.

In some embodiments, the air flow generator 802 includes a variable speed air pump the air pressure from which can be controlled directly without the use of a valve 804.

The bioamplifier 818 amplifies and processes signals from the detection system 824 and further converts signals from the detection system 824 from one format (e.g., analog) to another format (e.g., digital) to be provided to the main controller 820 for further processing and analysis.

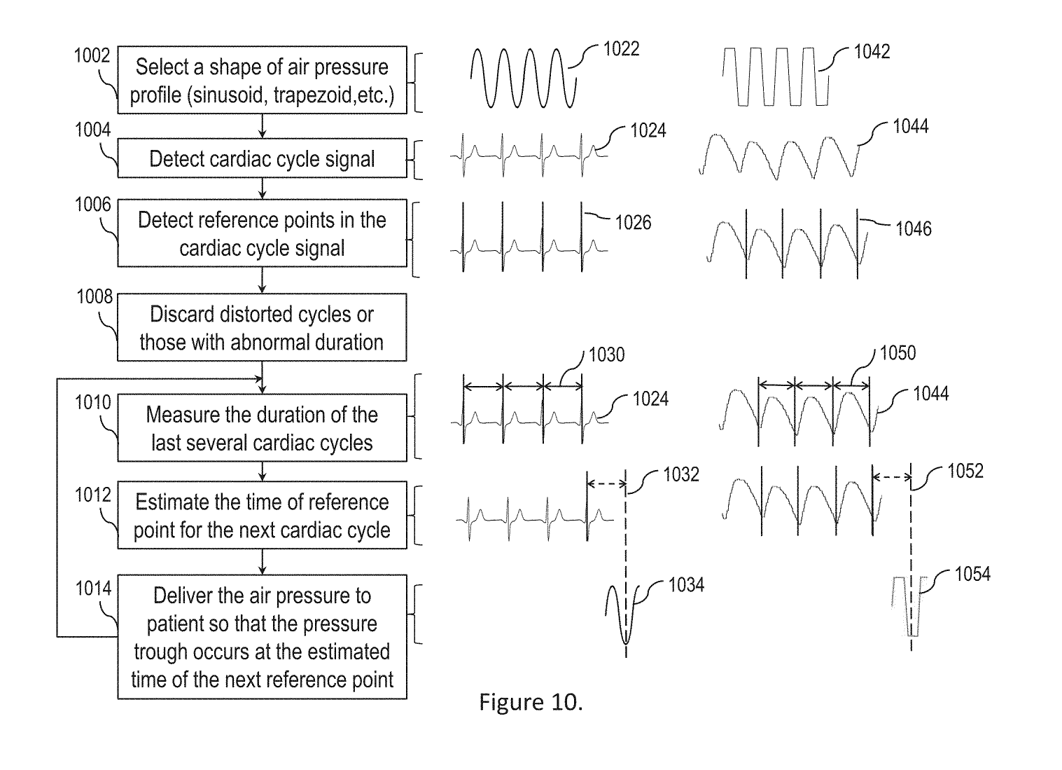

The main controller 820 has multiple functions. The main controller 820 receives the signals indicating cardiac cucles (e.g., ECG triggers) from the detection system 824 and the input from the clinician regarding the shape of the air pressure profile, and the maximum and minimum airway pressure levels. Alternatively, the airway pressure levels can be adjusted in response to the detected apneas and hypopneas. The main controller 820 then controls the valve 804 or the air pump speed of the air pressure generator 801 to cycle the airway pressure level between the maximum and the minimum. The main controller 820 estimates a reference point for the timing of the next cardiac cycle based on the last several detected cardiac cycles and delivers minimum airway pressure at the reference point to synchronize airway pressure with the estimated cardiac cycle, such as described below in conjunction with FIG. 10.

Referring to FIG. 9, a line 902 shows a pressure signal from a nasal cannula. A line 906 shows an ECG trace of a patient to demonstrate that several heart beats occur per breath cycle. A line 904 shows changes in the airway pressure of a BiPAP therapy, in which pressure relieve to the lungs coincides with exhalation and not with the cardiac cycle. Arrows 910 show the cardiac cycles impacted by the high intrathoracic pressure due to BiPAP. The length of an arrow 910 reflects the magnitude of the BiPAP impact on the cardiac cycle. A line 908 shows one embodiment of the cardio-adaptive PAP therapy delivering pressure relief in the shape of a sine wave trough during the systole of the ventricles, therefore reducing the negative impact of NPPV on the venous return, cardiac output, pulmonary arteries, and the workload of the right ventricle. The pressure relief coincides with the heart rate.

Referring to FIG. 10, the flow diagram shows the steps of a method for determining the timing of applied pressure of the cardio-adaptive PAP therapy. At 1002, the main controller 820 receives a user selection of a shape of an air pressure profile. The selection may be made, for example, by a manufacturer or a health care provider, and, is preselected or preset from the patient's viewpoint. The shape may be, for example, sinusoidal, trapezoidal, triangular, square, rectangular or the like. For simplicity and clarity, FIG. 10 illustrates a sinusoid profile 1022 and a trapezoid profile 1042. At 1004, the sensors 816 detects a cardiac cycle signal. Cardiac cycles 1024 are detected with sensors 816 that include electrocardiography (ECG) sensors. Cardiac cycles 1044 are detected with sensors 816 that include photoplethysmography (PPG) sensors. Cardiac cycles 1024 and cardiac cycles 1044 are shown for the sinusoid profile 1022 and the trapezoid profile 1042, respectively. At 1006, the main controller 820 detects reference points in the cardiac cycle. The reference points, for instance, can be aligned with the R-peak of the electrocardiography signal, or the minima of the photoplethysmography signal. Reference points 1026 and 1046 are shown for the electrocardiography signal 1024 and for the photoplethysmography signal 1044, respectively. At 1008, the main controller 820 discards distorted cycles or those with abnormal duration. The two examples of the ECG and PPG signals do not contain any abnormal cycles and this step is not illustrated. The main controller 820 may use a filter to discard any reference point that is too close or too far from the previous reference point. For example, this may occur when the detected duration between the reference points is not physiological or due to arrhythmia. At 1010, the main controller 820 measures the duration of the last several cardiac cycles. Durations 1030 and 1050 are shown for the electrocardiography signal 1024 and for the photoplethysmography signal 1044, respectively. At 1012, the main controller 820 estimates, for example by extrapolating, the timing of a reference point for the next cardiac cycle. Reference points 1032 and 1052 are shown for the electrocardiography signal 1024 and for the photoplethysmography signal 1044, respectively. In some embodiments, in order to estimate the timing of the next cardiac cycle, several cardiac cycles are detected, and reference points are selected within the cycles (R-peaks in the ECG signal, for instance). Once the distorted cycles (due to electrical noise, for instance) or the cycles of abnormal duration are discarded, the duration of the remaining cycles is determined. The duration of the remaining cycles can be averaged to determine the estimate for the timing of the reference point of the next cycle. Alternatively, a trend analysis of the duration of the last several cycles can be performed in order to estimate the duration of the next cycle more accurately and therefore to determine the timing of the next reference point more precisely. In patients with highly pronounced respiratory sinus arrhythmia (i.e., variability of heart rate within a breath cycle), the duration of the last several cardiac cycles can be correlated with the position of the cardiac cycle within the breath cycle. The estimated timing of the next reference point of the cardiac cycle then is adjusted to reflect its position within the ongoing breath cycle.

At 1014, the main controller 820 provides a control signal to the air flow controller 810 for delivering the air pressure to the patient via the air flow generator 802 so the pressure trough occurs, or substantially occurs within 10% of the cardiac cycle duration with the estimated reference point. A pressure trough 1034 and a pressure trough 1054 are shown for the sinusoid profile 1022 and the trapezoid profile 1042, respectively. The process continues and repeats 1010, 1012, and 1014 until an intervening event occurs, such as power off or pressure termination command to the main controller 820.

Estimating of the timing of the next cardiac cycle is performed because an airflow generator is not fast enough to be triggered off the last detected cardiac cycle.

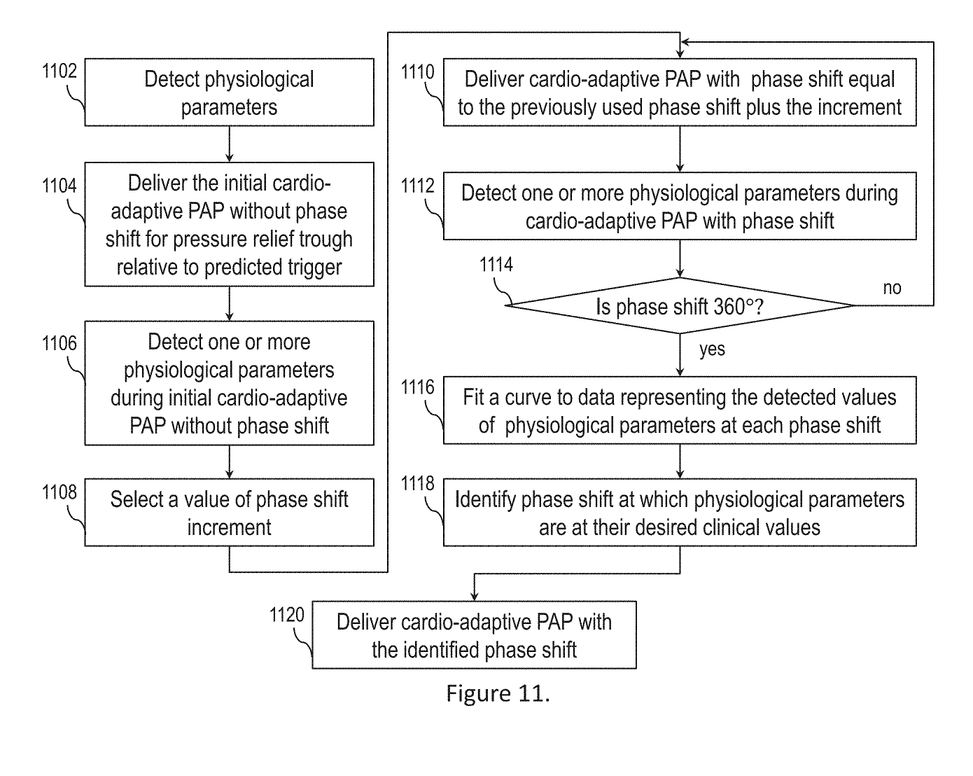

Referring to FIG. 11, the flow diagram shows the steps of a method for calibrating the cardio-adaptive PAP therapy for a patient. Because the mask volume, the length of the hose and the tubing inside an NPPV device and the airway size, lung size and lung distensibility and the chest wall and diaphragm compliance of the patient vary, the time of the pressure relief troughs of cardio-adaptive PAP therapy may require calibration. Calibration may be performed once for each patient, or once at the beginning of each therapy sessions, or periodically throughout a therapy session. Such a calibration can be achieved by first detecting, specific physiological parameters of a patient (such as systemic arterial blood pressure, cardiac output, augmentation index of an arterial pulse wave, pulmonary artery pressure, etc.) and then shifting the timing of the pressure relief troughs to achieve an improvement in the detected physiologic parameter (for instance, a reduction in systemic blood pressure or an increase in the cardiac output, etc). By gradually varying the timing shift of the pressure relief troughs and then detecting a physiological parameter a relationship between the timing shift and the values of the detected physiological parameters can be identified for a specific patient. From the identified relationship, the timing shift that produces the desired value of the physiological parameter (for instance, the lowest systolic blood pressure) can be established. Once the timing shift is established, it can be used in a specific patient for the duration of the cardio-adaptive PAP therapy. Alternatively, this timing shift may be periodically recalibrated and adjusted. Furthermore, the levels of the minimum and the maximum pressure settings as well as the shape of the pressure relief curve can be modified for a specific patient by monitoring other physiological parameters (such blood oxygen saturation, end tidal carbon dioxide, heart rate variability as a measure of the balance between the sympathetic and parasympathetic nervous systems). If the cardio-adaptive PAP therapy is administered during sleep, other physiological parameters (such as apnea-hypopnea index, arousal index, flow limitations) can be used to adjust the minimal and maximal pressure levels and the shape of the pressure relief curve. At 1102, the detection system 824 detects physiological parameters of the patient. At 1104, the air pressure system 801 delivers an initial cardio-adaptive airway pressure profile to the patient via the mask 814. At 1106, the detection system 824 detects one or more physiological parameters during the initial cardio-adaptive airway pressure profile delivery to the patient. At 1108, the main controller 820 sets an increment for the subsequent time shifts (typically 15° or 30° degree phase from the next reference point). At 1110, the air pressure system 801 delivers a cardio-adaptive airway pressure profile with timing shift equal to the previous timing shift plus the set increment to the patient. At 1112, the detection system 824 detects one or more physiological parameters of the patient during the cardio-adaptive airway pressure profile with a time shift delivery to the patient. The steps 1110 and 1112 are repeated until the timing shift corresponds to a phase shift of 360° at 1114. At 1116, the main controller 820 then fits a curve to the data the representing the detected values of physiological parameters at each phase shift. The curve represents the relationship between one or more physiological parameters and the timing shifts of the cardio-adaptive airway pressure profiles delivered to the patient. At 1116, the main controller 820 identifies the timing shift corresponding to the desired value of the physiological parameter (for instance, the lowest blood pressure or the highest cardiac output, etc.). At 1120, the air pressure system 801 delivers a cardio-adaptive airway pressure profile with timing shift identified in 1118. If a periodic recalibration is used, the recalibration procedure does not need to use the whole range of the timing shifts (i.e. phase shift from 0° to 360°), but may focus on the timing shifts surrounding the previously identified timing shift. For instance, if the timing shift corresponding to 30° was used prior to recalibration, the recalibration procedure may step through the timing shifts corresponding to phase shifts from 0° to 60° for instance.

In some patient subgroups, such as patients with moderate sleep apnea who are otherwise healthy, no calibration maybe needed. In such patients, the relatively healthy heart appears to shift its cardiac cycle and to adjust to the pressure relief troughs delivered by the cardio-adaptive PAP therapy system.

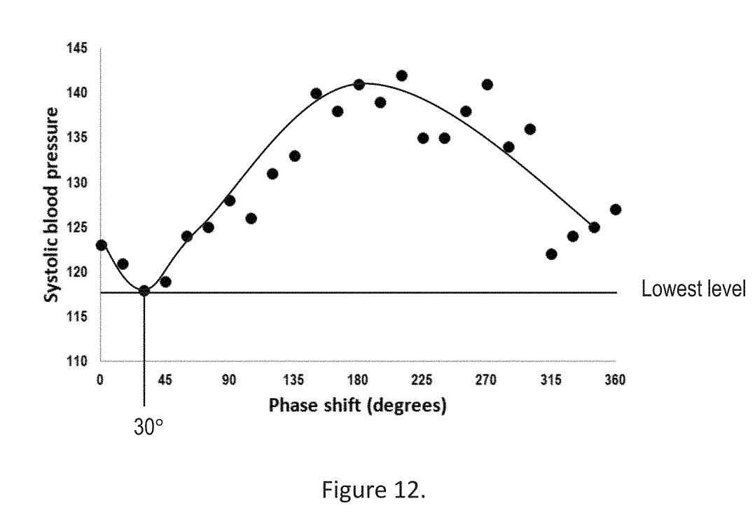

Referring to FIG. 12, the graph shows the relationship between the detected physiological parameter systolic blood pressure and the timing shift of the pressure relief troughs of the cardio-adaptive PAP therapy. The systolic blood pressure was detected for a specific patient via a beat-to-beat volume-clamp method and the timing shifts were varied in increments corresponding to the phase shifts from 0° to 360°. The increment was set to 15°. The graph illustrates that, for that specific patient, the cardio-adaptive PAP with the pressure relief trough at the phase shift of 30° results in the lowest systolic blood pressure. In practice, the 30° phase shift would then be used for this patient to administer the cardio-adaptive PAP.

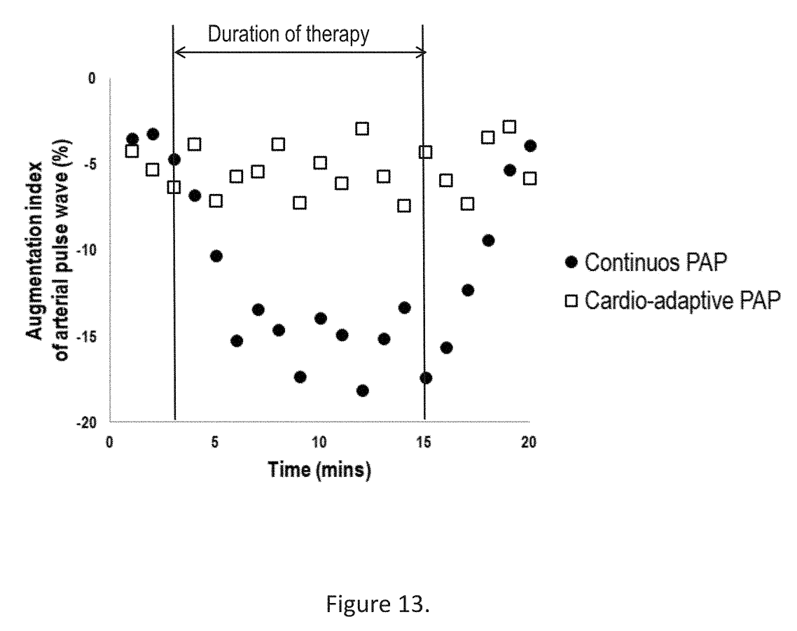

Referring to FIG. 13, acute effects of continuous PAP and cardio-adaptive PAP were recorded in a healthy subject while awake. After 10 minutes of rest in supine position, 3 minutes of baseline data (arterial pulse wave) were recorded with an infrared photoplethysmography (PPG) sensor. CPAP at 10 cmH2O pressure was then administered for 12 minutes, followed by additional 5 minutes of recording. The subject continued to rest in supine position for additional 20 minutes (a washout period between treatments). Three minutes of baseline data (arterial pulse wave) were recorded again. Cardio-adaptive PAP with peak pressures of 15 cmH2O and trough pressures of 5 cmH2O was then administered for 12 minutes, followed by additional 5 minutes of recording. The graph shows that the augmentation index decreased with the CPAP treatment and remained largely unaffected with the cardio-adaptive PAP treatment. While not a direct measure of hemodynamics, the augmentation index of arterial index during the acute application of PAP therapy is thought to reflect the acute changes in cardiac output. The data therefore can be interpreted as the CPAP causing an acute reduction in cardiac output and the cardio-adaptive PAP having a minimal acute effect on cardiac output.

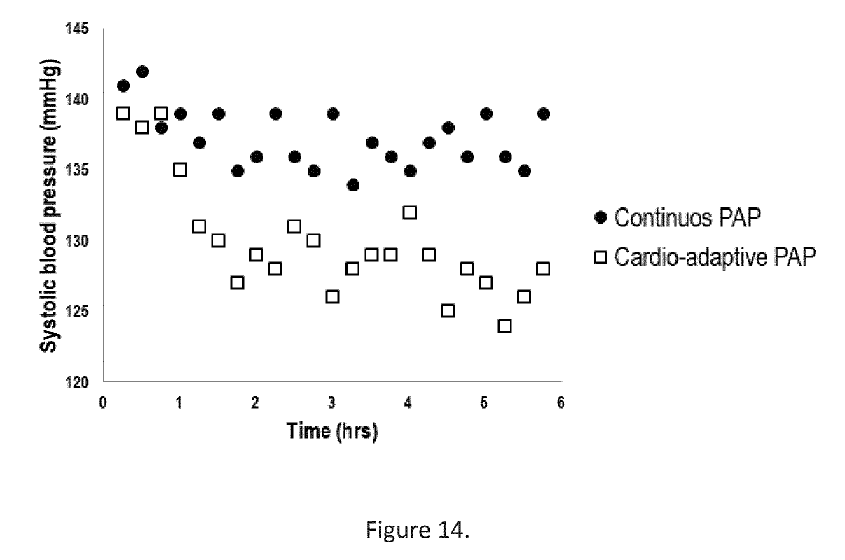

Referring to FIG. 14, overnight effects of continuous PAP and cardio-adaptive PAP were recorded in a patient with moderate sleep apnea accustomed to wearing a full-face mask. A non-invasive beat-to-beat blood pressure monitor was used to monitor the effects on the systolic blood pressure. On the first night, only CPAP at 10 cmH20 pressure was then administered. On the second night, only cardio-adaptive PAP with peak pressures of 15 cmH2O and trough pressures of 5 cmH2O was then administered. The graph shows that the systolic blood pressure decreased slightly with the CPAP treatment. With the cardio-adaptive PAP treatment the systolic blood pressure decreased significantly. Systolic blood pressure is a direct measure of hemodynamics and the cardio-adaptive PAP causes significant improvement in overnight systolic blood pressure and is therefore likely to significantly improve the hemodynamics of patients with sleep apnea in the long term.

The above description illustrates various embodiments of the present disclosure along with examples of how aspects of the particular embodiments may be implemented. The above examples should not be deemed to be the only embodiments, and are presented to illustrate the flexibility and advantages of the particular embodiments as defined by the following claims. Based on the above disclosure and the following claims, other arrangements, embodiments, implementations and equivalents may be employed without departing from the scope of the present disclosure as defined by the claims.

What is claimed is:

1. A cardio-adaptive non-invasive positive airway pressure device comprising:
an airflow generator to provide pressurized air to a human;
a detector to detect a cardiac cycle of the human; and
a control unit to estimate a next cardiac cycle based on the detected cardiac cycle and to provide a control signal to the air flow generator to control timing of the providing of the pressurized air to the human based on the estimated cardiac cycle.

2. The device of claim 1 wherein the control unit selects a reference point in the detected cardiac cycle, determines a duration of a plurality of detected cardiac cycles, estimates a reference point for a next cardiac cycle, and sets the estimated reference point as a timing parameter for the control signal.

3. The device of claim 2 wherein the control unit sets the timing parameters so that a pressure trough of the pressurized air substantially occurs with the estimated reference point.

4. The device of claim 1 further comprising a valve timing the delivery of the provided pressurized air to the next cardiac cycle.

5. The device of claim 1 wherein the detector of a cardiac cycle is selected from a group of an arterial volume detection system (a photoplethysmography or a plethysmography system), an arterial pressure pulse wave detection system, an electrocardiography system, an acoustic heart beat detection system, and a ballistic heart beat detection system.

6. The device of claim 1 wherein the detector of a cardiac cycle is a remote system that is not in direct contact with the patient.

7. A method for determining cardio-adaptive positive airway pressure, the method comprising:
detecting cardiac cycle of a human;
estimating a next cardiac cycle; and
determining timing parameters of pressurized air based on the estimated cardiac cycle.

8. The method of claim 7 further comprising providing pressurized air to the human according to the timing parameters.

9. The method of claim 7 wherein estimating a next cardiac cycle comprises:
selecting a reference point in the detected cardiac cycle;
determining a duration of a plurality of detected cardiac cycles; and
estimating a reference point for a next cardiac cycle;
wherein determining timing parameters of pressurized air based on the estimated cardiac cycle comprises setting the estimated reference point for the timing parameters for providing pressurized air to the human.

10. The method of claim 7 further comprising providing pressurized air to the human according to the timing parameters so that a pressure trough of the pressurized air substantially occurs with the estimated reference point.

11. The method of claim 7 further comprising periodically adjusting the timing parameters of the pressurized air during the treatment period.

12. The method of claim 7 further comprising:
detecting one or more physiological parameters before pressurized air is delivered to the human;
delivering, during an initial time period, pressurized air having a cardio-adaptive airway pressure profile to the human;
detecting one or more physiological parameters during the initial time period;
comparing one or more physiological parameters before and during the initial time period;
adjusting the parameters of the airway pressure profile; and
delivering the pressurized air having the adjusted airway pressure profile to the human.

13. The method of claim 12 wherein the detected physiological parameter is selected from a group of systemic arterial pressure, pulmonary arterial pressure, cardiac output, systemic vascular resistance, and pulmonary vascular resistance.

14. The method of claim 12 wherein the detected physiological parameters are selected from a group of contour characteristics of the arterial pulse wave, variability of arterial pulse wave amplitude, shape characteristics of the echocardiogram, autonomous nervous system status, frequency components of the heart rate variability frequency spectrum, and the frequency components of the arterial pulse wave frequency spectrum.

15. The method of claim 12 wherein contour characteristics of an arterial pulse wave is an augmentation index.

16. The method of claim 12 wherein contour characteristics of an arterial pulse wave is the maximal slope of the ascending systolic portion or descending diastolic portion of the pulse wave.

17. The method of claim 12 wherein the shape characteristic of electrocardiogram or the contour characteristic of arterial pulse wave is a ratio of the duration of systole to the duration of diastole.

18. The method of claim 12 wherein detecting cardiac cycle of a human comprises:
detecting airway pressure; and
determining cardiac cycle based on the detected airway pressure.

19. A method for determining a timing parameter of pressurized air, the method comprising:
selecting a reference point in a detected cardiac cycle;
determining a duration of a plurality of detected cardiac cycles; and
estimating a reference point for a next cardiac cycle for setting the timing parameter to provide pressurized air to the human.
